(19) United States
(12) Patent Application Publication
(10) Pub. No.: US 2005/0221453 A1
(43) Pub. Date: Oct. 6, 2005

(54) L-cysteine producing microorganism and method for producing L-cysteine

(75) Inventors: Hiroshi Takagi, Fukui-shi (JP); Shigeru Nakamori, Yoshida-gun (JP); Akihito Yamaguchi, Ibaraki-shi (JP); Kunihiko Nishino, Ibaraki-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: AJINOMOTO CO., INC, Tokyo (JP)

(21) Appl. No.: 10/957,828

(22) Filed: Oct. 5, 2004

(30) Foreign Application Priority Data
Mar. 31, 2004 (JP) 2004-103652

(51) Int. Cl. C12P 13/12; C12P 13/06; C12N 1/21

(52) U.S. Cl. 435/113; 435/252.33

(57) ABSTRACT

L-Cysteine is produced by culturing a microorganism having an ability to produce L-cysteine and modified so that expression of emrAB, emrKY, yojIH, acrEF, bcr, or cusA gene should be enhanced in a medium to produce and accumulate L-cysteine in the medium and collecting the L-cysteine from the medium. Genes coding for novel L-cysteine-excreting proteins are identified, and utilized for breeding of L-cysteine-producing microorganism to provide a novel method of producing L-cysteine.

### BACKGROUND OF THE INVENTION

1. Field of the Invention

The present invention relates to a method for producing L-cysteine. In more detail, the present invention relates to a microorganism suitable for the production of L-cysteine and a method for producing L-cysteine utilizing such a microorganism. L-Cysteine and L-cysteine derivatives are used in the fields of drugs, cosmetics and foods.

2. Description of the Related Art

L-Cysteine is conventionally obtained by extraction from keratin-containing substances such as hairs, horns and feathers or by microbial enzyme-catalyzed conversion of a precursor, DL-2-aminothiazoline-4-carboxylic acid. It has also been planned to produce L-cysteine in a large scale by an immobilized-enzyme method utilizing a novel enzyme.

Furthermore, it has been also attempted to produce L-cysteine by fermentation utilizing a microorganism. There is known a method for producing L-cysteine by using a microorganism in which cysteine metabolism is deregulated by means of DNA coding for serine acetyltransferase (EC 2.3.1.30, also referred to as “SAT” hereinafter) mutant that has a particular mutation which reduces feedback inhibition by L-cysteine (WO97/15673). Further, FEMS Microbiol. Lett., vol. 179, pp.453-459 (1999) discloses a method for producing L-cysteine by using Escherichia coli in which a gene coding for an SAT isozyme derived from Arabidopsis thaliana which is not subject to feedback inhibition by L-cysteine is introduced. Moreover, JP11-56381A discloses a method for producing L-cysteine using a microorganism overexpressing a gene coding for a protein which can excrete an antibiotic or a substance toxic to a microorganism directly from a cell.

Furthermore, the inventors of the present invention disclosed a method for producing L-cysteine by using a microorganism belonging to the genus Escherichia in which L-cysteine-decomposing pathway is suppressed and feedback inhibition of SAT by L-cysteine is reduced (JP11-155571A and JP2003-169668A). In these references, as means for suppressing the L-cysteine-decomposing pathway, reduction of intracellular cysteine desulfhydrase activity is disclosed.

Japanese Patent No. 2992010 disclosed a method for producing L-cysteine by using a microorganism in which expression of excretion genes such as mar gene is enhanced. In addition, it was disclosed in J. Bacteriol., 185, (2003) pp.1161-1166 that yfiK promoted excretion of L-cysteine. Furthermore, it was disclosed in J. Biol. Chem., 277 (2002) pp.49841-49849 that CydDC was involved in excretion of L-cysteine.

emrAB, emrKY, yojIh, acrEF, bcr and cusA genes were known as genes imparting resistances to various kinds of drugs to host microorganisms when they were overexpressed (J. Bacteriol., Vol. 183, (2001) pp.5803-5812). However, it has not been elucidated whether these genes have an ability to excrete L-cysteine.

### SUMMARY OF THE INVENTION

An object of the present invention is to identify a gene coding for a novel L-cysteine-excreting protein and utilize it for breeding of L-cysteine-producing bacteria, and to provide a novel method of producing L-cysteine.

The inventors of the present invention assiduously studied in order to achieve the aforementioned object. As a result, they found that L-cysteine can be produced in a marked amount by using a strain in which expression of genes coding for proteins with L-cysteine-excreting ability, specifically, emrAB, emrKY, yojIH, acrEF, bcr or cusA gene, is enhanced, and thereby they accomplished the present invention.

Thus, the present invention provides the followings.
(1) A microorganism having an ability to produce L-cysteine and modified so that expression of emrAB gene should be enhanced.
(2) The microorganism according to (1), wherein the emrAB gene is a gene defined in the following (A) or (B):
(A) a gene coding for a protein having the amino acid sequence of SEQ ID NO: 2 and a protein having the amino acid sequence of SEQ ID NO: 4, or
(B) a gene coding for a protein which exhibits 80% or more homology with a protein having the amino acid sequence of SEQ ID NO: 2 and has an ability to excrete L-cysteine, and a protein which exhibits 80% or more homology with a protein having the amino acid sequence of SEQ ID NO: 4 and has an ability to excrete L-cysteine.
(3) The microorganism according to (1), wherein the emrAB gene is a gene defined in the following (a) or (b):
(a) a gene having the nucleotide sequence of SEQ ID NO: 1 and the nucleotide sequence of SEQ ID NO: 3, or
(b) a gene comprising a gene hybridizable with a polynucleotide having the nucleotide sequence of SEQ ID NO: 1 under a stringent condition and coding for a protein having an ability to excrete L-cysteine and a gene hybridizable with a polynucleotide having the nucleotide sequence of SEQ ID NO: 3 under a stringent condition and coding for a protein having an ability to excrete L-cysteine.
(4) A microorganism having an ability to produce L-cysteine and modified so that expression of emrKY gene should be enhanced.
(5) The microorganism according to (4), wherein the emrKY gene is a gene defined in the following (C) or (D):
(C) a gene coding for a protein having the amino acid sequence of SEQ ID NO: 6 and a protein having the amino acid sequence of SEQ ID NO: 8, or
(D) a gene coding for a protein which exhibits 80% or more homology with a protein having the amino acid sequence of SEQ ID NO: 6 and has an ability to excrete L-cysteine, and a protein which exhibits 80% or more homology with a protein having the amino acid sequence of SEQ ID NO: 8 and has an ability to excrete L-cysteine.
(6) The microorganism according to (4), wherein the emrKY gene is a gene defined in the following (c) or (d):
(c) a gene having the nucleotide sequence of SEQ ID NO: 5 and the nucleotide sequence of SEQ ID NO: 7, or
(d) a gene comprising a gene hybridizable with a polynucleotide having the nucleotide sequence of SEQ ID NO: 5 under a stringent condition and coding for a protein having an ability to excrete L-cysteine and a gene hybridizable with a polynucleotide having the nucleotide sequence of SEQ ID NO: 7 under a stringent condition and coding for a protein having an ability to excrete L-cysteine.
(7) A microorganism having an ability to produce L-cysteine and modified so that expression of yojIH gene should be enhanced.
(8) The microorganism according to (7), wherein the yojIH gene is a gene defined in the following (E) or (F):
(E) a gene coding for a protein having the amino acid sequence of SEQ ID NO: 10 and a protein having the amino acid sequence of SEQ ID NO: 12, or
(F) a gene coding for a protein which exhibits 80% or more homology with a protein having the amino acid sequence of SEQ ID NO: 10 and has an ability to excrete L-cysteine, and a protein which exhibits 80% or more homology with a protein having the amino acid sequence of SEQ ID NO: 12 and has an ability to excrete L-cysteine.
(9) The microorganism according to (7), wherein the yojIH gene is a gene defined in the following (e) or (f):
(e) a gene having the nucleotide sequence of SEQ ID NO: 9 and the nucleotide sequence of SEQ ID NO: 11, or
(f) a gene comprising a gene hybridizable with a polynucleotide having the nucleotide sequence of SEQ ID NO: 9 under a stringent condition and coding for a protein having an ability to excrete L-cysteine and a gene hybridizable with a polynucleotide having the nucleotide sequence of SEQ ID NO: 11 under a stringent condition and coding for a protein having an ability to excrete L-cysteine.
(10) A microorganism having an ability to produce L-cysteine and modified so that expression of acrEF gene should be enhanced.
(11) The microorganism according to (10), wherein the acrEF gene is a gene defined in the following (G) or (H):
(G) a gene coding for a protein having the amino acid sequence of SEQ ID NO: 14 and a protein having the amino acid sequence of SEQ ID NO: 16, or
(H) a gene coding for a protein which exhibits 80% or more homology with a protein having the amino acid sequence of SEQ ID NO: 14 and has an ability to excrete L-cysteine, and a protein which exhibits 80% or more homology with a protein having the amino acid sequence of SEQ ID NO: 16 and has an ability to excrete L-cysteine.
(12) The microorganism according to (10), wherein the acrEF gene is a gene defined in the following (g) or (h):
(g) a gene having the nucleotide sequence of SEQ ID NO: 13 and the nucleotide sequence of SEQ ID NO: 15, or
(h) a gene comprising a gene hybridizable with a polynucleotide having the nucleotide sequence of SEQ ID NO: 13 under a stringent condition and coding for a protein having an ability to excrete L-cysteine and a gene hybridizable with a polynucleotide having the nucleotide sequence of SEQ ID NO: 15 under a stringent condition and coding for a protein having an ability to excrete L-cysteine.
(13) A microorganism having an ability to produce L-cysteine and modified so that expression of bcr gene should be enhanced.
(14) The microorganism according to (13), wherein the bcr gene is a gene defined in the following (I) or (J):
(I) a gene coding for a protein having the amino acid sequence of SEQ ID NO: 18, or
(J) a gene coding for a protein which exhibits 80% or more homology with a protein having the amino acid sequence of SEQ ID NO: 18 and has an ability to excrete L-cysteine.
(15) The microorganism according to (13), wherein the bcr gene is a gene defined in the following (i) or (j):
(i) a gene having the nucleotide sequence of SEQ ID NO: 17, or
(j) a gene comprising a gene hybridizable with a polynucleotide having the nucleotide sequence of SEQ ID NO: 17 under a stringent condition and coding for a protein having an ability to excrete L-cysteine.
(16) A microorganism having an ability to produce L-cysteine and modified so that expression of cusA gene should be enhanced.
(17) The microorganism according to (16), wherein the cusA gene is a gene defined in the following (K) or (L):
(K) a gene coding for a protein having the amino acid sequence of SEQ ID NO: 20, or
(L) a gene coding for a protein which exhibits 80% or more homology with a protein having the amino acid sequence of SEQ ID NO: 20 and has an ability to excrete L-cysteine.
(18) The microorganism according to (16), wherein the cusA gene is a gene defined in the following (k) or (l):
(k) a gene having the nucleotide sequence of SEQ ID NO: 19, or
(l) a gene comprising a gene hybridizable with a polynucleotide having the nucleotide sequence of SEQ ID NO: 19 under a stringent condition and coding for a protein having an ability to excrete L-cysteine.
(19) The microorganism according to any one of (1) to (18), which belongs to the genus Escherichia.
(20) The microorganism according to (19), which is Escherichia coli.
(21) The microorganism according to any one of (1) to (20), which is further modified so that serine acetyltransferase activity should be enhanced.
(22) A method for producing L-cysteine, which comprises culturing the microorganism according to any one of (1) to
(21) in a medium to produce and accumulate L-cysteine in the medium and collecting the L-cysteine from the medium.

### DESCRIPTION OF THE PREFERRED EMBODIMENTS

The microorganism of the present invention is a microorganism having an ability to produce L-cysteine and modified so that expression of emrAB, emrKY, yojIH, acrEF, bcr, or cusA gene should be enhanced. The microorganism of the present invention may be one obtained by modifying a microorganism having an ability to produce L-cysteine so that expression of the aforementioned genes should be enhanced, or one obtained by imparting an ability to produce L-cysteine to a microorganism in which expression of the aforementioned genes is enhanced. In the microorganism of the present invention, expression of two or more kinds of genes among the aforementioned genes may be enhanced.

In the present invention, the ability to produce L-cysteine means an ability of the microorganism of the present invention to accumulate L-cysteine in a medium in such an amount that the L-cysteine can be collected from the medium when the microorganism is cultured in the medium. In the present invention, the ability to produce L-cysteine may be imparted by modifying a parent strain with gene recombination technique or mutagenesis treatment. Further, a microorganism originally having an ability to produce L-cysteine may also be used. In the present invention, the term L-cysteine includes reduced type of L-cysteine and L-cystine, unless otherwise specified.

Examples of the method for imparting the ability to produce L-cysteine include methods utilizing mutagenesis treatment, genetic recombination technique and so forth. Examples of the mutagenesis treatment include, for example, a method of treating a microorganism with ultraviolet irradiation or a mutation-inducing agent used for ordinary mutagenesis treatment such as N-methyl-N′-nitro-N-nitrosoguanidine (NTG) or nitrous acid and selecting a mutant strain that has gained an ability to produce L-cysteine. Examples of the genetic recombination techniques include a method of enhancing the activity of serine acetyltransferase by genetic recombination as described below.

The microorganism of the present invention is preferably a microorganism belonging to the genus Escherichia. As a microorganism belonging to the genus Escherichia, those mentioned in Neidhardt et al. (Neidhardt, F. C. et al., Escherichia coli and Salmonella Typhimurium, American Society for Microbiology, Washington D.C., 1208, Table 1), for example, Escherichia coli and so forth, can be utilized. Examples of wild type strains of Escherichia coli include, for example, Escherichia coli K12 strain and derivatives thereof, Escherichia coli MG1655 strain (ATCC No. 47076), Escherichia coli W3110 strain (ATCC No. 27325) and so forth. These strains can be obtained from American Type Culture Collection (ATCC, Address: 12301 Parklawn Drive, Rockville, Md. 20852, United States of America).

In the present invention, emrAB gene refers to a gene containing emrA gene and emrB gene. Expressions of these genes may be simultaneously enhanced, or may be separately enhanced. Examples of the emrA gene include a gene coding for a protein having the amino acid sequence of SEQ ID NO: 2. Examples of the emrB gene include a gene coding for a protein having the amino acid sequence of SEQ ID NO: 4. These genes may be genes each coding for a protein having the amino acid sequence of SEQ ID NO: 2 or NO: 4 including substitution, deletion, insertion or addition of one or several amino acid residues, so long as they code for a protein having an ability to excrete L-cysteine. The number of amino acid residues meant by the aforementioned term “several” is preferably 2 to 20, more preferably 2 to 10, particularly preferably 2 to 5.

The ability to excrete L-cysteine can be measured by determining, when a microorganism in which the aforementioned gene is introduced is cultured in a medium, whether the amount of L-cysteine excreted in the medium is increased or not compared with the amount observed with a wild type strain.

emrA gene and emrB gene may also be genes each coding for a protein exhibiting 80% or more, preferably 90% or more, more preferably 95% or more homology with a protein having the amino acid sequence of SEQ ID NO: 2 or NO: 4, so long as they code for a protein having an ability to excrete L-cysteine. In the present invention, the degree of homology can be evaluated by known calculation methods such as BLAST search, FASTA search and CrustalW. BLAST (Basic Local Alignment Search Tool) is the heuristic search algorithm employed by the programs blastp, blastn, blastx, megablast, tblastn, and tblastx; these programs ascribe significance to their findings using the statistical methods of Karlin, Samuel and Stephen F. Altschul (Proc. Natl. Acad. Sci. USA, 1990, 87:2264-68; Proc. Natl. Acad. Sci. USA, 1993, 90:5873-7). FASTA search method described by W. R. Pearson (Methods in Enzymology, 1990 183:63- 98). ClustalW method described by Thompson J. D., Higgins D. G. and Gibson T. J. (Nucleic Acids Res. 1994, 22:4673-4680).

Specifically, emrA gene may be a gene having the nucleotide sequence of SEQ ID NO: 1 and emrB gene may be a gene having the nucleotide sequence of SEQ ID NO: 3. These genes may be genes hybridizable with a polynucleotide having the nucleotide sequence of SEQ ID NO: 1 or NO: 3 under a stringent condition, so long as they code for a protein having an ability to excrete L-cysteine. Examples of the stringent condition referred to in the present invention include, for example, a condition of washing one time, preferably two or three times, at salt concentrations of 1×SSC and 0.1% SDS, preferably 0.1×SSC and 0.1% SDS, at 60° C. after hybridization.

In the present invention, emrKY gene refers to a gene containing emrK gene and emrY gene. Expressions of these genes may be simultaneously enhanced, or may be separately enhanced. Examples of emrK gene include a gene coding for a protein having the amino acid sequence of SEQ ID NO: 6. Examples of emrY gene include a gene coding for a protein having the amino acid sequence of SEQ ID NO: 8. These genes may be genes each coding for a protein having the amino acid sequence of SEQ ID NO: 6 or NO: 8 including substitution, deletion, insertion or addition of one or several amino acid residues, so long as they code for a protein having an ability to excrete L-cysteine. They may also be genes each coding for a protein exhibiting 80% or more, preferably 90% or more, more preferably 95% or more homology with a protein having the amino acid sequence of SEQ ID NO: 6 or NO: 8, so long as they code for a protein having an ability to excrete L-cysteine. The number of amino acid residues meant by the aforementioned term “several” is preferably 2 to 20, more preferably 2 to 10, particularly preferably 2 to 5.

Specifically, emrK gene may be a gene having the nucleotide sequence of SEQ ID NO: 5 and emrY gene may be a gene having the nucleotide sequence of SEQ ID NO: 7. These genes may be genes hybridizable with a polynucleotide having the nucleotide sequence of SEQ ID NO: 5 or NO: 7 under a stringent condition, so long as they code for a protein having an ability to excrete L-cysteine.

In the present invention, yojIH gene refers to a gene containing yojI gene and yojH gene. Expressions of these genes may be simultaneously enhanced, or may be separately enhanced. Examples of yojI gene include a gene coding for a protein having the amino acid sequence of SEQ ID NO: 10. Examples of yojH gene include a gene coding for a protein having the amino acid sequence of SEQ ID NO: 12. These genes may be genes each coding for a protein having the amino acid sequence of SEQ ID NO: 10 or NO: 12 including substitution, deletion, insertion or addition of one or several amino acid residues, so long as they code for a protein having an ability to excrete L-cysteine. They may also be genes each coding for a protein exhibiting 80% or more, preferably 90% or more, more preferably 95% or more homology with a protein having the amino acid sequence of SEQ ID NO: 10 or NO: 12, so long as they code for a protein having an ability to excrete L-cysteine. The number of amino acid residues meant by the aforementioned term “several” is preferably 2 to 20, more preferably 2 to 10, particularly preferably 2 to 5.

Specifically, yojI gene include a gene having the nucleotide sequence of SEQ ID NO: 9 and yojH gene may be a gene having the nucleotide sequence of SEQ ID NO: 11. These genes may be genes hybridizable with a polynucleotide having the nucleotide sequence of SEQ ID NO: 9 or NO: 11 under a stringent condition, so long as they code for a protein having an ability to excrete L-cysteine.

In the present invention, acrEF gene refers to a gene containing acrE gene and acrf gene. Expressions of these genes may be simultaneously enhanced, or may be separately enhanced. Examples of acrE gene include a gene coding for a protein having the amino acid sequence of SEQ ID NO: 14. Examples of acrf gene include a gene coding for a protein having the amino acid sequence of SEQ ID NO: 16. These genes may be genes each coding for a protein having the amino acid sequence of SEQ ID NO: 14 or NO: 16 including substitution, deletion, insertion or addition of one or several amino acid residues, so long as they code for a protein having an ability to excrete L-cysteine. They may also be genes each coding for a protein exhibiting 80% or more, preferably 90% or more, more preferably 95% or more homology with a protein having the amino acid sequence of SEQ ID NO: 14 or NO: 16, so long as they code for a protein having an ability to excrete L-cysteine. The number of amino acid residues meant by the aforementioned term “several” is preferably 2 to 20, more preferably 2 to 10, particularly preferably 2 to 5.

Specifically, acrE gene may be a gene having the nucleotide sequence of SEQ ID NO: 13 and acrF gene may be a gene having the nucleotide sequence of SEQ ID NO: 15. These genes may be genes hybridizable with a polynucleotide having the nucleotide sequence of SEQ ID NO: 13 or NO: 15 under a stringent condition, so long as they code for a protein having an ability to excrete L-cysteine.

In the present invention, examples of bcr gene include a gene coding for a protein having the amino acid sequence of SEQ ID NO: 18. This gene may be a gene coding for a protein having the amino acid sequence of SEQ ID NO: 18 including substitution, deletion, insertion or addition of one or several amino acid residues, so long as it codes for a protein having an ability to excrete L-cysteine. It may also be a gene coding for a protein exhibiting 80% or more, preferably 90% or more, more preferably 95% or more homology with a protein having the amino acid sequence of SEQ ID NO: 18, so long as it codes for a protein having an ability to excrete L-cysteine. The number of amino acid residues meant by the aforementioned term “several” is preferably 2 to 20, more preferably 2 to 10, particularly preferably 2 to 5.

Specifically, bcr gene may be a gene having the nucleotide sequence of SEQ ID NO: 17. This gene may be a gene hybridizable with a polynucleotide having the nucleotide sequence of SEQ ID NO: 17 under a stringent condition, so long as it codes for a protein having an ability to excrete L-cysteine.

In the present invention, examples of cusA gene include a gene coding for a protein having the amino acid sequence of SEQ ID NO: 20. This gene may be a gene coding for a protein having the amino acid sequence of SEQ ID NO: 20 including substitution, deletion, insertion or addition of one or several amino acid residues, so long as it codes for a protein having an ability to excrete L-cysteine. It may also be a gene coding for a protein exhibiting 80% or more, preferably 90% or more, more preferably 95% or more homology with a protein having the amino acid sequence of SEQ ID NO: 20, so long as it codes for a protein having an ability to excrete L-cysteine. The number of amino acid residues meant by the aforementioned term “several” is preferably 2 to 20, more preferably 2 to 10, particularly preferably 2 to 5.

Specifically, cusA gene may be a gene having the nucleotide sequence of SEQ ID NO: 19. This gene may be a gene hybridizable with a polynucleotide having the nucleotide sequence of SEQ ID NO: 19 under a stringent condition, so long as it codes for a protein having an ability to excrete L-cysteine.

Hereafter, a method for enhancing expression of the emrAB gene will be explained. Expression of the other genes can also be enhanced in a similar manner.

The modification for enhancing expression of emrAB gene can be attained by, for example, increasing copy number of the emrAB gene in the cells of a microorganism by means of a genetic recombination technique. For example, a recombinant DNA can be prepared by ligating a DNA fragment containing emrAB gene to a vector functioning in a host microorganism, preferably a multi-copy vector, and used to transform the host microorganism. A plasmid comprising both of emrA gene and emrB gene may be used, or the emrA gene and emrB gene may be introduced by separate plasmids.

When emrAB gene of Escherichia coli is used, the emrAB gene can be obtained by polymerase chain reaction (PCR, refer to White, T. J. et al., Trends Genet. 5, 185 (1989)) using primers prepared on the basis of the nucleotide sequences shown in SEQ ID NOS: 1 and 3, and chromosomal DNA of Escherichia Coli as a template. The emrAB gene of other microorganisms can also be obtained from chromosomal DNA or chromosomal DNA library of those microorganisms by the hybridization method using a probe prepared on the basis of the aforementioned sequence. The chromosomal DNA can be prepared from a microorganism serving as a DNA donor by, for example, the method of Saito and Miura (refer to H. Saito and K. Miura, Biochem. Biophys. Acta, 72, 619 (1963); Text for Bioengineering Experiments, Edited by the Society for Bioscience and Bioengineering, Japan, pp.97-98, Baifukan, 1992).

Then, the obtained emrAB gene is ligated to a vector DNA that can function in the cells of host microorganism to prepare a recombinant DNA. Examples of the vector that can function in the cells of host microorganism include vectors autonomously replicable in the cells of host microorganism. Examples of the vectors autonomously replicable in the cells of Escherichia coli include pUC19, pUC18, pHSG299, pHSG399, pHSG398, pACYC184, (PHSG and pACYC are obtainable from Takara Bio), RSF1010, pBR322, pMW219 (pMW is obtainable from NIPPON GENE). In order to introduce a recombinant DNA prepared as described above into a microorganism, conventional transformation methods can be employed. For example, a method of treating recipient cells with calcium chloride so as to increase the permeability of the cells for DNA, which has been reported for Escherichia coli K-12 (Mandel, M. and Higa, A., J. Mol. Biol., 53, 159 (1970)), is available.

Increasing copy number of emrAB gene can also be attained by introducing multiple copies of the emrAB gene into a chromosomal DNA of a microorganism. Multiple copies of the emrAB gene may be introduced into a chromosomal DNA of a microorganism by homologous recombination technique in which a sequence multiply present on chromosomal DNA is targeted. A repetitive DNA or inverted-repeat present at the end of a transposable element may be used as the sequence multiply present on a chromosomal DNA. Alternatively, as disclosed in JP02-109985, emrAB gene may be incorporated into a transposon and, by transferring the transposon, multiply introduced into a chromosomal DNA.

Besides the gene amplification method explained above, expression of emrAB gene can also be enhanced by replacing an expression regulatory sequence such as a promoter of the emrAB gene with a stronger one on a chromosomal DNA or a plasmid. For example, lac promoter, trp promoter, trc promoter can be mentioned as strong promoters. Furthermore, a promoter of emrAB gene can also be modified to be stronger by introducing substitution of several nucleotides into the promoter region of the emrAB gene. Modification of an expression regulatory sequence can be combined with increasing the copy number of emrAB gene. Expression of emrAB gene may also be enhanced by amplifying an activator of expression of emrAB, or by deleting or attenuating a suppressor of expression of emrAB.

Hereafter, as the method for imparting an ability to produce L-cysteine to a microorganism, a method for enhancing an activity of L-cysteine biosynthetic enzyme will be explained. Enhancement of an activity of L-cysteine biosynthetic enzyme can be attained by, for example, enhancing serine acetyltransferase (SAT) activity. Enhancement of the SAT activity in cells of a microorganism can be attained by increasing copy number of a gene coding for SAT. For example, a recombinant DNA can be prepared by ligating a gene fragment coding for SAT into a vector that functions in a microorganism, preferably a multi-copy vector, and the recombinant DNA can be used to transform a host microorganism.

As SAT gene, a gene of a microorganism belonging to the genus Escherichia as well as genes of other organisms can be used. As a gene coding for SAT of Escherichia coli, cycE gene has been cloned from a wild strain and an L-cysteine-excretion mutant strain, and the nucleotide sequence thereof has been disclosed (Denk, D. and Boeck, A., J. General Microbiol., 133, 515-525 (1987)). Therefore, a SAT gene can be obtained by PCR utilizing primers prepared based on the nucleotide sequence of SAT (SEQ ID NO: 21) and chromosomal DNA of Escherichia coli as a template (refer to JP11-155571A). Genes coding for SAT of other microorganisms can also be obtained in a similar manner. Expression of the SAT gene obtainable as described above can be enhanced in the same manner as explained above for emrAB gene.

When a suppressing mechanism such as “feedback inhibition by L-cysteine” exists in the expression of SAT gene, expression of SAT gene can also be enhanced by modifying an expression regulatory sequence or a gene involved in the suppression so that SAT gene should become insensitive to the suppression mechanism.

SAT activity in cells of a microorganism can be further increased by making the microorganism carry mutant type SAT of which feedback inhibition by L-cysteine is reduced or eliminated. Examples of the mutant type SAT include SAT having a mutation which replaces an amino acid residue corresponding to the 256th methionine residue of a wild-type SAT (SEQ ID NO: 22) with an amino acid residue other than lysine residue and leucine residue, or a deletion which deletes a region of an amino acid residues corresponding to the 256th methionine residue and thereafter in a wild-type SAT. The amino acid residue other than lysine residue and leucine residue include 17 kinds of amino acid residues among the amino acids constituting ordinary proteins except for methionine residue, lysine residue and leucine residue. More preferred are isoleucine residue and glutamic acid residue. As a method of introducing a desired mutation into a wild-type SAT gene, site-specific mutagenesis can be mentioned. As a mutant type SAT gene, a mutant type cysE gene coding for a mutant type SAT of Escherichia coli is known (refer to WO97/15673 and JP11-155571A). Escherichia coli JM39-8 strain which harbors a plasmid pCEM256E containing a mutant type cysE gene coding for a mutant type SAT in which 256th methionine residue is replaced with a glutamic acid residue (E. coli JM39-8(pCEM256E), private number: AJ13391) was deposited at the National Institute of Bioscience and Human-Technology, Agency of Industrial Science and Technology (currently, National Institute of Advanced Industrial Science and Technology, Postal code: 305-8566, Central 6, 1-1-1 Higashi, Tsukuba-shi, Ibaraki-ken, Japan) on Nov. 20, 1997 and given an accession number of FERM P-16527. Then, the deposit was converted to an international deposit under the provisions of the Budapest Treaty on Jul. 8, 2002, and received an accession number of FERM BP-8112.

In the present invention, although “SAT which is insensitive to feedback inhibition by L-cysteine” may be one modified so that it should become insensitive to the feedback inhibition by L-cysteine, it may also be SAT originally free from the feedback inhibition by L-cysteine. For example, SAT of Arabidopsis thaliana is known to be free from the feedback inhibition by L-cysteine and can be suitably used for the present invention. As a plasmid containing the SAT gene derived from Arabidopsis thaliana, pEAS-m is known (FEMS Microbiol. Lett., 179 (1999) 453-459).

L-Cysteine can be efficiently and stably produced by culturing the microorganism of the present invention as described above in a suitable medium to produce and accumulate L-cysteine in the culture and collecting the L-cysteine from the culture. L-cysteine produced by the method of the present invention includes cystine in addition to reduced type of L-cysteine.

Medium used for culturing the microorganism may be ordinary medium containing carbon source, nitrogen source, sulfur source, inorganic ions, and the medium may further contain other organic components as required. As the carbon source, saccharides such as glucose, fructose, sucrose, molasses and starch hydrolysate, organic acids such as fumaric acid, citric acid and succinic acid may be used. As the nitrogen source, inorganic ammonium salts such as ammonium sulfate, ammonium chloride and ammonium phosphate, organic nitrogen such as soybean hydrolysate, ammonia gas, aqueous ammonia may be used. As the sulfur source, inorganic sulfur compounds such as sulfates, sulfites, sulfides, hyposulfites and thiosulfates may be used. It is preferable to add auxotrophic substances such as vitamin B₁, yeast extract and so forth in appropriate amounts as organic nutrients. Other than these, potassium phosphate, magnesium sulfate, iron ions, manganese ions and so forth may be added in small amounts if necessary.

Culture is preferably performed under an aerobic condition for 30 to 90 hours. Culture temperature is preferably controlled to be at 25° C. to 37° C., and pH is preferably controlled to be 5 to 8 during cultivation. For pH adjustment, inorganic or organic acidic or alkaline substances, ammonia gas and so forth may be used. Collection of L-cysteine from the culture may be attained by, for example, a combination of ordinary ion-exchange resin method, precipitation and other known methods.

### EXAMPLES

Hereafter, the present invention will be explained more specifically with reference to the following examples.

### (1) Construction of Strains Eith Enhanced L-cysteine Biosynthesis by Amplifying L-cysteine Excretion Genes

As a parent strain, the JM39 strain (F+ cysE51 tfr-8, Denk, D. and Bock, A., J. Gen. Microbiol., 133, 515-525 (1987)) was used. The JM39 strain was transformed with each of plasmids obtained by incorporating various cysteine excretion genes into pUC118 (pUCemrAB, pUCemrKY, pUCyojIH, pUCacrEF, pUCbcr, pUCcusA). The plasmids were constructed by the method described in J. Bacteriol., Vol. 183, 2001, 5803-5812, Materials and Methods and Table 1. pUCemrAB is a plasmid containing SalI-BamHI fragment of 3.9 kb containing emrR, emrA and emrB genes of Escherichia coli. pUCemrKY is a plasmid containing SphI-BamHI fragment of 7.5 kb containing evgS/A, emrK and emrY genes of Escherichia coli. pUCyojIH is a plasmid containing SalI-SphI fragment of 4.0 kb containing the yojI and yojH genes of Escherichia coli. pUCacrEF is a plasmid containing SalI-SphI fragment of 5.9 kb containing the envR, acrE and acrF genes of Escherichia coli. pUCbcr is a plasmid containing AccI-KpnI fragment of 2.3 kb containing the yeiD and bcr genes of Escherichia coli. pUCcusA is a plasmid containing SphI-EcoRI fragment of 9.0 kb containing the cusS, cusR/C/F/B and cusA genes of Escherichia coli. The transformants were selected on the basis of ampicillin resistance. The obtained transformants were further transformed with a plasmid containing a mutant type SAT gene in which the 256th Met was replaced with Ile (pACYC256I). The transformants were selected on the basis of both ampicillin resistance and chloramphenicol resistance. pACYC256I was constructed as follows from pCEM256I (JP11-155571A). That is, pCEM256I was digested with BamHI and SalI, and the excised fragment containing Met256Ile mutant type SAT gene (including the promoter region) was ligated to pACYC184 (NIPPON GENE) digested with the same restriction enzymes and thus pACYC256I was obtained.

### (2) Production of L-Cys (Reduced Type of L-cysteine)+L-CysH (L-cystine)

Each of the obtained transformants was plated on LB plate (10 g/L of trypton, 5 g/L of yeast extract, 5 g/L of NaCl, pH 7.0 and 15 g/L of agar) containing 50 mg/L of ampicillin and 100 mg/L of chloramphenicol, cultured at 37° C. for 12 to 24 hours, then inoculated into 20 mL of Cys-production medium (30 g/L of glucose, 10 g/L of NH₄Cl, 2 g/L of KH₂PO₄, 1 g/L of MgSO₄.7H₂O, 10 mg/L of FeSO₄.7H₂O, 10 mg/L of MnCl₂.4H₂O, 15 g/L of thiosulfuric acid and 50 mg/L of ampicillin (added every 24 hours), 100 mg/L of chloramphenicol, 20 g/L of CaCO₃) contained in a flask, and cultured at 30° C. for 24, 48 or 72 hours with shaking. Amount of the accumulated L-cysteine (L-Cys and L-CysH) was quantified by a bioassay using Leuconostoc mesenteroides (Tsunoda T. et al., Amino acids, 3, 7-13 (1961)) for each culture broth diluted with 0.5 N HCl in order to dissolve the precipitated L-cystine. The results are shown in Table 1.

TABLE 1

| | Accumulation of | | |
|---|---|---|---|
| | L-Cys + L-CysH (g/L) | | |
| Introduced plasmid | 24 hours | 48 hours | 72 hours |
| pACYC256I, pUC118 | 0.07 | 0.08 | 0.04 |
| pACYC256I, pUCemrAB | 0.25 | 0.34 | 0.30 |
| pACYC256I, pUCemrKY | 0.06 | 0.34 | 0.52 |
| pACYC256I, pUCyojIH | 0.07 | 0.20 | 0.34 |
| pACYC256I, pUCacrEF | 0.08 | 0.25 | 0.31 |
| pACYC256I, pUCbcr | 0.08 | 0.65 | 0.53 |
| pACYC256I, pUCcusA | 0.26 | 0.58 | 0.41 |

The results shown in Table 1 indicate that the accumulation of L-cysteine was markedly enhanced by the introduction of plasmids containing various cysteine excretion genes.

While the invention has been described in detail with reference to preferred embodiments thereof, it will be apparent to one skilled in the art that various changes can be made, and equivalents employed, without departing from the scope of the invention. Each of the aforementioned documents, including the foreign priority document, JP 2004-103652, is incorporated by reference herein in its entirety.

1. A microorganism having an ability to produce L-cysteine and modified so that expression of enrAB gene should be enhanced.

2. The microorganism according to claim 1, wherein the emrAB gene is a gene defined in the following (A) or (B):
(A) a gene coding for a protein having the amino acid sequence of SEQ ID NO: 2 and a protein having the amino acid sequence of SEQ ID NO: 4, or
(B) a gene coding for a protein which exhibits 80% or more homology with a protein having the amino acid sequence of SEQ ID NO: 2 and has an ability to excrete L-cysteine, and a protein which exhibits 80% or more homology with a protein having the amino acid sequence of SEQ ID NO: 4 and has an ability to excrete L-cysteine.

3. The microorganism according to claim 1, wherein the emrAB gene is a gene defined in the following (a) or (b):
(a) a gene having the nucleotide sequence of SEQ ID NO: 1 and the nucleotide sequence of SEQ ID NO: 3, or
(b) a gene comprising a gene hybridizable with a polynucleotide having the nucleotide sequence of SEQ ID NO: 1 under a stringent condition and coding for a protein having an ability to excrete L-cysteine and a gene hybridizable with a polynucleotide having the nucleotide sequence of SEQ ID NO: 3 under a stringent condition and coding for a protein having an ability to excrete L-cysteine.

4. A microorganism having an ability to produce L-cysteine and modified so that expression of emrKY gene should be enhanced.

5. The microorganism according to claim 4, wherein the emrKY gene is a gene defined in the following (C) or (D):
(C) a gene coding for a protein having the amino acid sequence of SEQ ID NO: 6 and a protein having the amino acid sequence of SEQ ID NO: 8, or
(D) a gene coding for a protein which exhibits 80% or more homology with a protein having the amino acid sequence of SEQ ID NO: 6 and has an ability to excrete L-cysteine, and a protein which exhibits 80% or more homology with a protein having the amino acid sequence of SEQ ID NO: 8 and has an ability to excrete L-cysteine.

6. The microorganism according to claim 4, wherein the emrKY gene is a gene defined in the following (c) or (d):
(c) a gene having the nucleotide sequence of SEQ ID NO: 5 and the nucleotide sequence of SEQ ID NO: 7, or
(d) a gene comprising a gene hybridizable with a polynucleotide having the nucleotide sequence of SEQ ID NO: 5 under a stringent condition and coding for a protein having an ability to excrete L-cysteine and a gene hybridizable with a polynucleotide having the nucleotide sequence of SEQ ID NO: 7 under a stringent condition and coding for a protein having an ability to excrete L-cysteine.

7. A microorganism having an ability to produce L-cysteine and modified so that expression of yojIH gene should be enhanced.

8. The microorganism according to claim 7, wherein the yojIH gene is a gene defined in the following (E) or (F):
(E) a gene coding for a protein having the amino acid sequence of SEQ ID NO: 10 and a protein having the amino acid sequence of SEQ ID NO: 12, or
(F) a gene coding for a protein which exhibits 80% or more homology with a protein having the amino acid sequence of SEQ ID NO: 10 and has an ability to excrete L-cysteine, and a protein which exhibits 80% or more homology with a protein having the amino acid sequence of SEQ ID NO: 12 and has an ability to excrete L-cysteine.

9. The microorganism according to claim 7, wherein the yojIH gene is a gene defined in the following (e) or (f):
(e) a gene having the nucleotide sequence of SEQ ID NO: 9 and the nucleotide sequence of SEQ ID NO: 11, or
(f) a gene comprising a gene hybridizable with a polynucleotide having the nucleotide sequence of SEQ ID NO: 9 under a stringent condition and coding for a protein having an ability to excrete L-cysteine and a gene hybridizable with a polynucleotide having the nucleotide sequence of SEQ ID NO: 11 under a stringent condition and coding for a protein having an ability to excrete L-cysteine.

10. A microorganism having an ability to produce L-cysteine and modified so that expression of acrEF gene should be enhanced.

11. The microorganism according to claim 10, wherein the acrEF gene is a gene defined in the following (G) or (H):
(G) a gene coding for a protein having the amino acid sequence of SEQ ID NO: 14 and a protein having the amino acid sequence of SEQ ID NO: 16, or
(H) a gene coding for a protein which exhibits 80% or more homology with a protein having the amino acid sequence of SEQ ID NO: 14 and has an ability to excrete L-cysteine, and a protein which exhibits 80% or more homology with a protein having the amino acid sequence of SEQ ID NO: 16 and has an ability to excrete L-cysteine.

12. The microorganism according to claim 10, wherein the acrEF gene is a gene defined in the following (g) or (h):
(g) a gene having the nucleotide sequence of SEQ ID NO: 13 and the nucleotide sequence of SEQ ID NO: 15, or
(h) a gene comprising a gene hybridizable with a polynucleotide having the nucleotide sequence of SEQ ID NO: 13 under a stringent condition and coding for a protein having an ability to excrete L-cysteine and a gene hybridizable with a polynucleotide having the nucleotide sequence of SEQ ID NO: 15 under a stringent condition and coding for a protein having an ability to excrete L-cysteine.

13. A microorganism having an ability to produce L-cysteine and modified so that expression of bcr gene should be enhanced.

14. The microorganism according to claim 13, wherein the bcr gene is a gene defined in the following (I) or (J):
(I) a gene coding for a protein having the amino acid sequence of SEQ ID NO: 18, or
(J) a gene coding for a protein which exhibits 80% or more homology with a protein having the amino acid sequence of SEQ ID NO: 18 and has an ability to excrete L-cysteine.

15. The microorganism according to claim 13, wherein the bcr gene is a gene defined in the following (i) or (i):
(i) a gene having the nucleotide sequence of SEQ ID NO: 17, or
(j) a gene comprising a gene hybridizable with a polynucleotide having the nucleotide sequence of SEQ ID NO: 17 under a stringent condition and coding for a protein having an ability to excrete L-cysteine.

16. A microorganism having an ability to produce L-cysteine cysteine and modified so that expression of cusA gene should be enhanced.

17. The microorganism according to claim 16, wherein the cusA gene is a gene defined in the following (K) or (L):
(K) a gene coding for a protein having the amino acid sequence of SEQ ID NO: 20, or
(L) a gene coding for a protein which exhibits 80% or more homology with a protein having the amino acid sequence of SEQ ID NO: 20 and has an ability to excrete L-cysteine.

18. The microorganism according to claim 16, wherein the cusA gene is a gene defined in the following (k) or (l):
(k) a gene having the nucleotide sequence of SEQ ID NO: 19, or
(l) a gene comprising a gene hybridizable with a polynucleotide having the nucleotide sequence of SEQ ID NO: 19 under a stringent condition and coding for a protein having an ability to excrete L-cysteine.

19. The microorganism according to any one of claims 1 to 18, which belongs to the genus Escherichia.

20. The microorganism according to claim 19, which is Escherichia coli.

21. The microorganism according to any one of claims 1 to 20, which is further modified so that serine acetyltransferase activity should be enhanced.

22. A method for producing L-cysteine comprising culturing the microorganism according to any one of claims 1 to 21 in a medium to produce and accumulate L-cysteine in the medium and collecting the L-cysteine from the medium.


SEQUENCE LISTING (22 sequences)

SEQ ID NO: 1 - DNA, 1173 bp, Escherichia coli
  CDS (1)..(1173)
atgagcgcaaatgcggagactcaaaccccgcagcaaccggtaaagaagagcggcaaacgtaagcgtctgctcctccttctcaccttgctctttataattattgccgtagcgatagggatttattggtttttggtactgcgtcacttcgaagaaaccgatgacgcatacgtggcagggaatcaaattcaaattatgtctcaggtgtctggcagcgtgacgaaagtctgggccgataacaccgattttgtaaaagaaggcgacgtgctggtcactctcgacccgacagatgctcgccaggcgtttgaaaaagccaaaactgcactggcttccagcgttcgccaaacccaccagctgatgattaacagcaagcagttgcaggcgaatattgaggtgcagaaaatcgccctcgcgaaagcacaaagcgactacaaccgccgtgtgccgctgggcaatgccaacctgattggtcgcgaagagctgcaacacgcccgcgacgccgtcaccagtgcccaggcgcaactggacgtcgcgattcaacaatacaatgccaatcaggcgatgattctggggactaaactggaagatcagccagccgtgcaacaggctgccaccgaagtacgtaacgcctggctggcgctggagcgtactcgtattatcagtccgatgaccggttatgtctcccgccgcgcggtacagcctggggcgcaaattagcccaacgacgccgctgatggcggtcgttccagccaccaatatgtgggtggatgccaactttaaagagacgcagattgccaatatgcgtatcggtcagccggtcactatcaccacggatatttacggcgatgatgtgaaatacaccggtaaagtggttggtctggatatgggcacaggtagcgcgttctcactgcttccagcgcaaaatgcgaccggtaactggatcaaagtcgttcagcgtctgcctgtgcgtatcgaactggaccagaaacagctggagcaatatccgctgcgtatcggtttgtccacgctggtgagcgtcaataccactaaccgtgacggtcaggtactggcaaataaagtacgttccactccggtagcggtaagcaccgcgcgtgaaatcagcctggcacctgtcaataaactgatcgacgatatcgtaaaagctaacgctggctaa

SEQ ID NO: 2 - protein, 390 aa, Escherichia coli
MSANAETQTPQQPVKKSGKRKRLLLLLTLLFIIIAVAIGIYWFLVLRHFEETDDAYVAGNQIQIMSQVSGSVTKVWADNTDFVKEGDVLVTLDPTDARQAFEKAKTALASSVRQTHQLMINSKQLQANIEVQKIALAKAQSDYNRRVPLGNANLIGREELQHARDAVTSAQAQLDVAIQQYNANQAMILGTKLEDQPAVQQAATEVRNAWLALERTRIISPMTGYVSRRAVQPGAQISPTTPLMAVVPATNMWVDANFKETQIANMRIGQPVTITTDIYGDDVKYTGKVVGLDMGTGSAFSLLPAQNATGNWIKVVQRLPVRIELDQKQLEQYPLRIGLSTLVSVNTTNRDGQVLANKVRSTPVAVSTAREISLAPVNKLIDDIVKANAG

SEQ ID NO: 3 - DNA, 1539 bp, Escherichia coli
  CDS (1)..(1539)
atgcaacagcaaaaaccgctggaaggcgcgcaactggtcattatgacgattgcgctgtcactggcgacattcatgcaggtgctggactccaccattgctaacgtggcgatccccactatcgccgggaatctgggctcatcgctcagccagggaacgtgggtaatcacttctttcggggtggcgaatgccatctcgatcccgcttaccggctggctggcaaagcgcgtcggggaagtgaaactgttcctttggtccaccatcgcctttgctattgcgtcgtgggcgtgtggtgtctccagcagcctgaatatgctgatcttcttccgcgtgattcaggggattgtcgccgggccgttgatcccgctttcgcaaagtctattgctgaataactacccgccagccaaacgctcgatcgcgctggcgttgtggtcgatgacggtgattgtcgcgccaatttgcggcccgatcctcggcggttatatcagcgataattaccactggggctggatattcttcatcaacgtgccgattggcgtggcggtggtgttgatgacactgcaaactctgcgcggacgtgaaacccgcaccgaacggcggcggattgatgccgtggggctggcactgctggttattggtatcggcagcctgcagattatgctcgaccgcggtaaagagctggactggttttcatcacaggaaattatcatccttaccgtggtggcggtggtggctatctgcttcctgattgtctgggagctgaccgacgataacccgatagtcgatctgtcgttgtttaagtcgcgcaacttcaccatcggctgcttgtgtatcagcctcgcgtatatgctctacttcggcgctattgttctgctgccgcagttgttgcaggaggtctacggttacacggcgacctgggcaggtttggcctctgcgccggtagggattattccggtgatcctgtcgccgattatcggccgcttcgcgcataaactggatatgcggcggctggtaaccttcagctttattatgtatgccgtctgcttctactggcgtgcctatacctttgaaccaggtatggattttggcgcgtcggcctggccgcagtttatccaggggtttgcggtggcctgcttctttatgccgctgaccaccattacgctgtctggtttgccaccggaacgactggcggcggcatcgagcctctctaactttacgcgaacgctggcggggtctatcggcacgtcgataaccacgaccatgtggaccaaccgcgagtcgatgcaccatgcgcagttgactgagtcggtaaacccgttcaacccgaatgcccaggcgatgtacagtcaactggaagggcttgggatgacgcaacagcaggcatcaggctggattgcccagcagatcaccaatcaggggctgattatttccgccaatgagatcttctggatgtcagccgggatattcctcgtcctgctggggctggtgtggtttgctaaaccgccatttggcgcaggtggcggcggaggcggtgcgcactaa

SEQ ID NO: 4 - protein, 512 aa, Escherichia coli
MQQQKPLEGAQLVIMTIALSLATFMQVLDSTIANVAIPTIAGNLGSSLSQGTWVITSFGVANAISIPLTGWLAKRVGEVKLFLWSTIAFAIASWACGVSSSLNMLIFFRVIQGIVAGPLIPLSQSLLLNNYPPAKRSIALALWSMTVIVAPICGPILGGYISDNYHWGWIFFINVPIGVAVVLMTLQTLRGRETRTERRRIDAVGLALLVIGIGSLQIMLDRGKELDWFSSQEIIILTVVAVVAICFLIVWELTDDNPIVDLSLFKSRNFTIGCLCISLAYMLYFGAIVLLPQLLQEVYGYTATWAGLASAPVGIIPVILSPIIGRFAHKLDMRRLVTFSFIMYAVCFYWRAYTFEPGMDFGASAWPQFIQGFAVACFFMPLTTITLSGLPPERLAAASSLSNFTRTLAGSIGTSITTTMWTNRESMHHAQLTESVNPFNPNAQAMYSQLEGLGMTQQQASGWIAQQITNQGLIISANEIFWMSAGIFLVLLGLVWFAKPPFGAGGGGGGAH

SEQ ID NO: 5 - DNA, 1056 bp, Escherichia coli
  CDS (1)..(1056)
atggaattagaagacatgattagtacagatgacgcctatgtcacggggaatgcagatccaatttctgcacaagtctcaggtagtgtcactgtcgttaatcataaagatacgaactacgttcgacaaggtgacattttagtttcactggataaaactgatgccactatcgcactcaataaagctaaaaataatctggcaaatattgttcggcaaacgaataaactatacttacaggataaacaatacagtgccgaagtcgcttcagcacgtattcagtatcaacaatctttagaagattataaccgtcgagtgccgttagcgaagcagggggttatttcaaaagaaacgctggagcataccaaagatacgttaataagtagcaaagcggcattgaatgccgctatccaggcttataaagcgaataaagctttagtaatgaacacaccattaaaccgtcagccacaagtcgttgaagcggcggatgcaactaaagaagcctggttggcgcttaaacgtacggatattaagagtccggttaccggctatattgcccagagaagtgttcaggtcggcgaaacagtgagccccggacaatcgttaatggctgtcgtaccggcacgtcaaatgtgggttaatgccaactttaaagaaacacaactcacggatgtacggattggtcaatcggtcaatattatcagcgatctttatggtgaaaatgttgtgtttcatggtcgggtgacagggatcaatatgggaaccggcaatgcgttctccttattacctgcacaaaatgcgacagggaactggatcaaaatcgttcagcgtgtaccggttgaagtttctcttgatccaaaagaactcatggaacaccccttgcgtattggtttatcgatgacagcaactattgatacgaagaacgaagacattgccgagatgcctgagctggcttcaaccgtgacctccatgccggcttataccagtaaggctttagttatcgataccagtccgatagaaaaagaaattagcaacattatttcgcataatggacaactttaa

SEQ ID NO: 6 - protein, 351 aa, Escherichia coli
MELEDMISTDDAYVTGNADPISAQVSGSVTVVNHKDTNYVRQGDILVSLDKTDATIALNKAKNNLANIVRQTNKLYLQDKQYSAEVASARIQYQQSLEDYNRRVPLAKQGVISKETLEHTKDTLISSKAALNAAIQAYKANKALVMNTPLNRQPQVVEAADATKEAWLALKRTDIKSPVTGYIAQRSVQVGETVSPGQSLMAVVPARQMWVNANFKETQLTDVRIGQSVNIISDLYGENVVFHGRVTGINMGTGNAFSLLPAQNATGNWIKIVQRVPVEVSLDPKELMEHPLRIGLSMTATIDTKNEDIAEMPELASTVTSMPAYTSKALVIDTSPIEKEISNIISHNGQL

SEQ ID NO: 7 - DNA, 1539 bp, Escherichia coli
  CDS (1)..(1539)
atggcaatcactaaatcaactccggcaccattaaccggtgggacgttatggtgcgtcactattgcattgtcattagcgacatttatgcaaatgttggattccactatttctaacgtcgcaataccgacaatatctggctttctgggagcatcaacagacgaaggcacctgggttatcacctcgtttggtgtagcaaatgccattgcgatccctgttactggcaggttggcacaaagaataggcgaattaagattatttttactttcagtcacttttttttcgctgtcttcattaatgtgtagcctatcgaccaatcttgatgtgctgatattttttagagtcgttcaggggttaatggcggggccgttaattccactgtcacagagtttattattaaggaattatccgccagaaaaaagaacatttgctctggcattatggtcaatgaccgtgattatcgctccgatatgtggaccgatattgggcggttatatttgtgataactttagctggggttggatatttttaatcaatgtccctatggggattatcgtcctgacattatgcttaaccttacttaaaggaagagaaactgagacttcaccggtcaaaatgaatctaccaggactgaccctgttagtgctcggtgttggtggcttgcaaattatgcttgataaagggcgcgatctggattggttcaactcgagtacaataataatattaacagtagtatcagttatttctctgatctctttagtcatttgggagtcgacctcagagaacccgattcttgatctcagtttgtttaagtcccgtaacttcaccattggtattgtgagtatcacatgcgcgtatttattttactctggagcgatcgtccttatgccgcagttactccaggaaacgatggggtataatgcgatatgggccggacttgcttatgcgcccatcggcatcatgccactattaatttcacctttgataggacgttatggcaacaaaatagacatgcggttgttagtgacatttagttttttgatgtatgcggtttgctattactggcgttctgtgacatttatgccaacgattgattttacaggcatcattttgccgcagttttttcagggattcgccgttgcctgtttctttttacccttaacaacgatttcgttttcaggcttgccagataataaatttgccaatgcctcgagtatgagtaatttttttcgtaccttgtcaggatcagttggtacgtcgttgacaatgacgctgtggggacgacgcgaatcgttacaccatagtcagttgacagcaaccatcgatcaatttaaccccgtgtttaattcatcgtcacaaattatggataaatattatggttcgctttcaggagttcttaatgaaattaataatgaaataacccagcagtcactttctatttctgcaaatgagattttccgtatggcggctattgcttttatcttacttacggttttggtttggtttgcgaaaccgccgtttacagcgaaaggcgttgggtga

SEQ ID NO: 8 - protein, 512 aa, Escherichia coli
MAITKSTPAPLTGGTLWCVTIALSLATFMQMLDSTISNVAIPTISGFLGASTDEGTWVITSFGVANAIAIPVTGRLAQRIGELRLFLLSVTFFSLSSLMCSLSTNLDVLIFFRVVQGLMAGPLIPLSQSLLLRNYPPEKRTFALALWSMTVIIAPICGPILGGYICDNFSWGWIFLINVPMGIIVLTLCLTLLKGRETETSPVKMNLPGLTLLVLGVGGLQIMLDKGRDLDWFNSSTIIILTVVSVISLISLVIWESTSENPILDLSLFKSRNFTIGIVSITCAYLFYSGAIVLMPQLLQETMGYNAIWAGLAYAPIGIMPLLISPLIGRYGNKIDMRLLVTFSFLMYAVCYYWRSVTFMPTIDFTGIILPQFFQGFAVACFFLPLTTISFSGLPDNKFANASSMSNFFRTLSGSVGTSLTMTLWGRRESLHHSQLTATIDQFNPVFNSSSQIMDKYYGSLSGVLNEINNEITQQSLSISANEIFRMAAIAFILLTVLVWFAKPPFTAKGVG

SEQ ID NO: 9 - DNA, 1644 bp, Escherichia coli
  CDS (1)..(1644)
atggaacttcttgtacttgtctggcggcagtatcgctggccatttatcagtgtgatggcgctaagcctcgccagtgcggcattaggcattggcttaattgcttttatcaatcagcgccttatcgaaacggcggataccagtctgctggtgttgccggagtttctgggattattgctgctgttgatggcagtcactctcggatcgcaactggcgctcaccactttggggcatcacttcgtttaccgactgcgtagtgaatttatcaagcggattctggatactcacgtcgagcgcattgaacaactcggtagcgcctcgttgctggcggggttaaccagcgatgtgcgcaatatcaccattgcttttgtgcgtctgccggaactggtgcaggggatcattctcactatcggttcagcggcgtatctgtggatgctgtcgggcaaaatgttgctggtaacggctatctggatggcgatcaccatctggggcggttttgtgctggtggcgcgggtgtacaaacatatggcgaccctgcgtgaaaccgaagacaagctgtacacggattttcaaactgtacttgaagggcgcaaagagctgactctgaaccgggaacgcgccgagtatgtgtttaacaacctctacattcctgatgcgcaagagtatcgccaccatattattcgcgcagacaccttccatcttagtgccgtgaactggtcaaacatcatgatgctgggcgcaatcggcctggtgttctggatggcgaacagcctcggttgggctgataccaacgttgccgcgacctattcgttgacgcttttattcctgcgtacgccgctgctttcggcggttggcgcattgccgacgctgctgacggcgcaggtggcgtttaacaagctgaacaaattcgcgctcgcgcctttcaaagcagagtttccgcgcccgcaggcgtttcccaactggcaaacgctggagctgcgtaacgtgacgtttgcttatcaggataacgcgttttccgttggtccgattaatctcaccatcaaacgtggcgagctgctgtttctgattggcggcaacggtagcggaaaatcgacgctggcgatgttgttgacgggcttgtatcagccacaaagcggcgaaatcttgctggatggcaaacctgtcagcggcgaacaaccggaagattatcgcaaactgttttcggcagtgtttaccgatgtctggctgtttgatcaactgctggggccggagggtaaacccgctaacccgcaactggttgagaagtggctggcgcagctgaaaatggctcataagcttgagttaagcaacgggcgtattgttaacctgaagttatcaaaagggcagaaaaaacgcgtggcgctgttgctggcgctggcagaagaacgcgatattatcctgctggatgaatgggcggcggatcaggatccacacttccgtcgtgagttttatcaggtgttgctgccgctgatgcaggagatgggtaaaactattttcgctatcagtcatgatgatcattactttatccacgccgaccgcctgctggaaatgcgcaatgggcaacttagcgagctgacgggcgaagagcgcgatgccgcttcgcgtgatgccgttgcccggacggcataa

SEQ ID NO: 10 - protein, 547 aa, Escherichia coli
MELLVLVWRQYRWPFISVMALSLASAALGIGLIAFINQRLIETADTSLLVLPEFLGLLLLLMAVTLGSQLALTTLGHHFVYRLRSEFIKRILDTHVERIEQLGSASLLAGLTSDVRNITIAFVRLPELVQGIILTIGSAAYLWMLSGKMLLVTAIWMAITIWGGFVLVARVYKHMATLRETEDKLYTDFQTVLEGRKELTLNRERAEYVFNNLYIPDAQEYRHHIIRADTFHLSAVNWSNIMMLGAIGLVFWMANSLGWADTNVAATYSLTLLFLRTPLLSAVGALPTLLTAQVAFNKLNKFALAPFKAEFPRPQAFPNWQTLELRNVTFAYQDNAFSVGPINLTIKRGELLFLIGGNGSGKSTLAMLLTGLYQPQSGEILLDGKPVSGEQPEDYRKLFSAVFTDVWLFDQLLGPEGKPANPQLVEKWLAQLKMAHKLELSNGRIVNLKLSKGQKKRVALLLALAEERDIILLDEWAADQDPHFRREFYQVLLPLMQEMGKTIFAISHDDHYFIHADRLLEMRNGQLSELTGEERDAASRDAVARTA

SEQ ID NO: 11 - DNA, 1473 bp, Escherichia coli
  CDS (1)..(1473)
atgaccatggtggagcgcctggagggtgtcgcgcaggagagttcgaacggctggaataacgccggaaccgggcattctgcactgatggaactgaactacaccccgcaaaacgccgatggcagcatcagtattgaaaaagcagtcgccattaacgaagcatttcagatttcccgccagttctgggcgcaccaggttgagcgcggcgtgctgcgtactccgcgttcatttatcaataccgttccgcatatgagctttgtctggggcgaggataacgtcaatttcctgcgcgcccgttacgccgcgttgcaacaaagctcgctgtttcgcggtatgcgttactctgaagatcacgcgcagatcaaagagtgggcaccgttagtgatggaagggcgcgatccgcaacagaaagtggcagccacgcgtacggaaattggtaccgatgtgaactacggcgagatcacccgccagttaattgcttccttgcagaagaaatctaacttctcgctgcaactcagcagcgaagtccgcgccctaaagcgtaatgacgataacacctggaccgttaccgttgccgatctgaaaaatggcactgcacagaacattcgtgccaaatttgtctttatcggcgcgggcggtgcggcgctgaagctgttacaggaatcggggattccggaagcgaaagactacgccggtttcccggtgggcggacagttccttgtttcggaaaacccggacgtggttaatcaccatctggcgaaggtttacggtaaagcatccgttggcgcaccaccgatgtcggttccgcatatcgatacccgcgttctggacggtaaacgcgtagtgctgtttgggccatttgccaccttctcaaccaaattcctcaaaaacggttcattgtgggatctaatgagttccaccaccacctctaacgtgatgccgatgatgcacgtcgggctggataatttcgatctggtgaaatatctggtgagtcaggtgatgttgagtgaagaggatcgttttgaagcgttgaaagagtactatccgcaggcgaaaaaagaggactggcgtttgtggcaagcggggcagcgcgtgcagattatcaagcgtgatgccgagaaaggtggcgtactgcgtctgggtactgaagtcgtcagtgaccagcaaggaaccattgccgcgctcctgggggcatcgccaggggcgtcaaccgccgcgccgattatgttgaatctgctggaaaaagtatttggcgatcgtgtttccagcccgcaatggcaggctacgttgaaagcgatcgttccgtcttatggacgcaagctgaacggtgatgtagcggcaacagaacgcgagttgcagtacaccagcgaagtgctggggttgaactacgacaagccgcaggcagcagatagtacgccgaaaccgcagttgaaaccgcaacccgttcaaaaagaagtggcggatattgcgttgtaa

SEQ ID NO: 12 - protein, 490 aa, Escherichia coli
MTMVERLEGVAQESSNGWNNAGTGHSALMELNYTPQNADGSISIEKAVAINEAFQISRQFWAHQVERGVLRTPRSFINTVPHMSFVWGEDNVNFLRARYAALQQSSLFRGMRYSEDHAQIKEWAPLVMEGRDPQQKVAATRTEIGTDVNYGEITRQLIASLQKKSNFSLQLSSEVRALKRNDDNTWTVTVADLKNGTAQNIRAKFVFIGAGGAALKLLQESGIPEAKDYAGFPVGGQFLVSENPDVVNHHLAKVYGKASVGAPPMSVPHIDTRVLDGKRVVLFGPFATFSTKFLKNGSLWDLMSSTTTSNVMPMMHVGLDNFDLVKYLVSQVMLSEEDRFEALKEYYPQAKKEDWRLWQAGQRVQIIKRDAEKGGVLRLGTEVVSDQQGTIAALLGASPGASTAAPIMLNLLEKVFGDRVSSPQWQATLKAIVPSYGRKLNGDVAATERELQYTSEVLGLNYDKPQAADSTPKPQLKPQPVQKEVADIAL

SEQ ID NO: 13 - DNA, 1158 bp, Escherichia coli
  CDS (1)..(1158)
atgacgaaacatgccaggtttttcctcctgccctcctttattctgatctccgcggctttaatcgccggttgtaacgataagggagaagagaaagctcacgtcggtgaaccgcaggttaccgttcatattgtaaaaacggccccgttagaagttaagactgaattaccaggccgcaccaatgcttatcgtatagccgaagttcgcccacaggttagcgggatcgtactgaatcgcaatttcactgaaggcagcgatgtgcaagcaggccagtccctgtaccagatcgatcccgcgacctatcaggcaaattatgacagcgcgaaaggcgaactggcgaaaagtgaagccgccgccgccatcgcgcatttgacggtaaaacgttacgttccgctcgtgggtacgaaatacatcagccagcaggagtacgaccaggccattgctgatgctcgtcaggccgatgccgccgtgattgccgcaaaagccacagtcgaaagcgctcgcatcaatcttgcttataccaaagtcactgcgccaattagcggacgtatcggcaaatcgactgtgaccgaaggcgctcttgtcactaatgggcaaacgactgaactggcgactgtccagcagctcgatcctatctacgttgatgtgacccaatccagcaacgattttatgaggctgaagcaatccgtagagcaaggaaatttgcataaggaaaacgccaccagcaacgtagagttggtcatggaaaacggtcaaacctatcccctgaaaggtacgctgcaattctccgatgtgaccgttgatgaaagcaccggctccataaccctacgtgctgtcttccctaacccgcaacatacgcttttgccgggtatgtttgtgcgtgcacggattgatgaaggcgtccaacctgacgccattcttatcccgcaacaaggcgttagccgcacaccgcgtggtgatgcaaccgtgctgattgttaacgataaaagtcaggttgaagcgcgccctgtcgttgccagtcaggcgattggcgataaatggttgattagtgaaggactgaaatctggcgatcaagtcattgtcagcggcctgcaaaaagcgcgtccgggagagcaggttaaagccactaccgatacccccgcagatactgcatcgaagtaa

SEQ ID NO: 14 - protein, 385 aa, Escherichia coli
MTKHARFFLLPSFILISAALIAGCNDKGEEKAHVGEPQVTVHIVKTAPLEVKTELPGRTNAYRIAEVRPQVSGIVLNRNFTEGSDVQAGQSLYQIDPATYQANYDSAKGELAKSEAAAAIAHLTVKRYVPLVGTKYISQQEYDQAIADARQADAAVIAAKATVESARINLAYTKVTAPISGRIGKSTVTEGALVTNGQTTELATVQQLDPIYVDVTQSSNDFMRLKQSVEQGNLHKENATSNVELVMENGQTYPLKGTLQFSDVTVDESTGSITLRAVFPNPQHTLLPGMFVRARIDEGVQPDAILIPQQGVSRTPRGDATVLIVNDKSQVEARPVVASQAIGDKWLISEGLKSGDQVIVSGLQKARPGEQVKATTDTPADTASK

SEQ ID NO: 15 - DNA, 3105 bp, Escherichia coli
  CDS (1)..(3105)
atggcaaacttttttattcgacgaccgatatttgcatgggtgctggccattattctgatgatggcgggcgcactggcgatcctacaattgcccgtcgctcagtatccaacaattgcaccgcctgcggtttctgtttcagcaaactatccgggcgctgatgcgcagaccgtgcaggatacggtgacgcaggttatcgaacagaatatgaacggtatcgataacctgatgtatatgtcctccaccagcgattccgccggtagcgtgacaattacccttaccttccagtccgggaccgatcctgatatcgcgcaagtgcaggtgcagaacaaactccagctcgccacgccgttgctgccgcaggaggttcagcagcaggggatcagtgttgaaaagtccagtagcagctatttgatggtggcgggctttgtctctgataacccaggcaccacacaggacgatatctcggactatgtggcctctaacgttaaagatacgcttagccgtctgaatggcgtcggtgacgtacagcttttcggcgcacagtatgcgatgcgtatctggctggatgccgatctgctaaacaaatataaactgacaccggttgatgtgattaaccagttgaaggtacagaacgatcagatcgctgccggacagttgggcggaacgccagcgttaccagggcaacaattgaacgcctcgattattgctcagacgcggtttaaaaatccggaagaattcggcaaagtgaccctgcgcgtaaacagtgacggctcggtggtacgcctgaaagatgtcgcacgggttgaacttggcggtgaaaactataacgttatcgctcgtatcaacggaaaaccggcggcgggcctggggattaagctggcaaccggcgcgaatgctctcgataccgcgaaagccattaaggcaaaactggcggaattacagccattcttcccgcagggaatgaaggttctctacccttatgacaccacgccattcgtccagctttctattcacgaagtggtaaaaacgctgttcgaagccattatgctggtgttcctggtgatgtatctgttcttgcagaatatgcgagcaacgctgatccccaccattgcggtacccgtggtgttgttagggacgtttgccatcctcgccgcttttggttactccatcaacacactaacgatgttcgggatggtgcttgccatcgggctgctcgtcgatgatgcgatagtggtggtggagaacgtcgagcgcgtgatgatggaggataagctcccgccaaaagaagcgacggaaaaatcgatgtcgcaaattcagggcgcactggtgggtatcgcgatggtgctgtcagcggtatttattccgatggcattcttcggcggttctactggggcaatttatcgccagttctctatcaccatcgtttcggcaatggcgctttctgttctggtggcattgattcttacccctgcgttatgtgcaacgctgcttaaacccgtctctgctgagcatcacgaaaataagggcggtttcttcggttggtttaataccaccttcgatcatagcgttaaccactacaccaacagcgtcggcaaaatcctcggatccacaggacgatatttactgatctatgcgctgattgttgcaggaatggtggtgttgtttttacgtcttccgtcttccttcttacctgaagaggatcagggtgtctttctgaccatgattcagttacccgctggcgcgacgcaagagcggacgcaaaaagtgttggatcaagttacggattactatctgaagaacgagaaagcgaacgttgaaagtgtctttacggttaacggctttagcttcagcggccaggcacaaaacgccggtatggccttcgtcagtctgaaaccgtgggaagagcgtaatggtgacgaaaacagtgcggaagcggtaatccatcgtgccaaaatggaattgggcaagatccgcgacggttttgtcattccattcaatatgccagccattgttgaactgggcacggcaacgggtttcgactttgagttaattgatcaggctgggctgggtcacgatgccctaacccaggcccgtaaccagttgcttggtatggcggcgcaacatcctgccagcttagtcagcgtgcgccctaatggcctggaagacaccgcgcagtttaaactggaagttgaccaggaaaaggcgcaggcattaggtgtttcactttctgacatcaatcagaccatttcaacggcgctgggtgggacttacgttaacgacttcatcgaccgtggccgcgtgaaaaagttgtatgttcaggcggatgccaaattccgtatgctgccagaagatgtcgataaactttatgtccgcagcgccaacggcgaaatggtgccattctcggcctttaccacttcacattgggtgtatggctctccgcgactggaacgctacaacggtctgccgtcaatggagattcagggggaagccgcgccaggaaccagttccggcgatgccatggcgttgatggaaaaccttgcgtcaaaattacctgcgggcattggttatgactggacgggtatgtcgtatcaggaacgcttatcgggaaaccaggctcccgctctggtagcaatttcctttgtggttgttttcctgtgccttgctgcactctatgaaagctggtcaattcctgtctcggttatgttggtagtgccgttagggattgtcggcgtgctgctggcggcgacactctttaatcaaaaaaatgacgtctactttatggtgggcttgctaacgacaattggcttgtcggccaaaaacgctattttgatcgttgagttcgctaaagatctcatggagaaagagggtaaaggtgttgttgaagcgacactgatggcagtacgtatgcgtctgcgtcctatcctgatgacctctctcgcctttattctcggcgtattaccgctagctatcagtaacggtgccggcagtggcgcgcagaacgctgtgggtatcggggtaatgggaggaatggtctctgcaacgttgctggcaatcttcttcgtaccggtgttctttgtggtgatccgccgttgctttaaaggataa

SEQ ID NO: 16 - protein, 1034 aa, Escherichia coli
MANFFIRRPIFAWVLAIILMMAGALAILQLPVAQYPTIAPPAVSVSANYPGADAQTVQDTVTQVIEQNMNGIDNLMYMSSTSDSAGSVTITLTFQSGTDPDIAQVQVQNKLQLATPLLPQEVQQQGISVEKSSSSYLMVAGFVSDNPGTTQDDISDYVASNVKDTLSRLNGVGDVQLFGAQYAMRIWLDADLLNKYKLTPVDVINQLKVQNDQIAAGQLGGTPALPGQQLNASIIAQTRFKNPEEFGKVTLRVNSDGSVVRLKDVARVELGGENYNVIARINGKPAAGLGIKLATGANALDTAKAIKAKLAELQPFFPQGMKVLYPYDTTPFVQLSIHEVVKTLFEAIMLVFLVMYLFLQNMRATLIPTIAVPVVLLGTFAILAAFGYSINTLTMFGMVLAIGLLVDDAIVVVENVERVMMEDKLPPKEATEKSMSQIQGALVGIAMVLSAVFIPMAFFGGSTGAIYRQFSITIVSAMALSVLVALILTPALCATLLKPVSAEHHENKGGFFGWFNTTFDHSVNHYTNSVGKILGSTGRYLLIYALIVAGMVVLFLRLPSSFLPEEDQGVFLTMIQLPAGATQERTQKVLDQVTDYYLKNEKANVESVFTVNGFSFSGQAQNAGMAFVSLKPWEERNGDENSAEAVIHRAKMELGKIRDGFVIPFNMPAIVELGTATGFDFELIDQAGLGHDALTQARNQLLGMAAQHPASLVSVRPNGLEDTAQFKLEVDQEKAQALGVSLSDINQTISTALGGTYVNDFIDRGRVKKLYVQADAKFRMLPEDVDKLYVRSANGEMVPFSAFTTSHWVYGSPRLERYNGLPSMEIQGEAAPGTSSGDAMALMENLASKLPAGIGYDWTGMSYQERLSGNQAPALVAISFVVVFLCLAALYESWSIPVSVMLVVPLGIVGVLLAATLFNQKNDVYFMVGLLTTIGLSAKNAILIVEFAKDLMEKEGKGVVEATLMAVRMRLRPILMTSLAFILGVLPLAISNGAGSGAQNAVGIGVMGGMVSATLLAIFFVPVFFVVIRRCFKG

SEQ ID NO: 17 - DNA, 435 bp, Escherichia coli
  CDS (1)..(435)
atgatggcgcggagaagtaatgcggtgcgaccactgaccatcatcagtcatcagcaccagaccggtggtatcaatatccaaccgccccgccgcatgcagtttccacgctaccggttcatcaagaaaatagagcaccgttgggtgatcagggtcgtccgtggagcaaacatagccctgaggcttattgagcatgaagtaacgtggaccgtgttgctgcgccacggggttgccatcgtaagcgacatcatgttcaggaagcagtttgaacgctgcattacggacgatttcgccatcgacggtgacacgattgccgcggatttcacgcccggcaatagcacggctaacgccgagttgctgtgcgataaatttatcaagtcgcatgtgtgtgattttgcctgtaaaaacggaggtcggggcattgacacgaaaatctga

SEQ ID NO: 18 - protein, 144 aa, Escherichia coli
MMARRSNAVRPLTIISHQHQTGGINIQPPRRMQFPRYRFIKKIEHRWVIRVVRGANIALRLIEHEVTWTVLLRHGVAIVSDIMFRKQFERCITDDFAIDGDTIAADFTPGNSTANAELLCDKFIKSHVCDFACKNGGRGIDTKI

SEQ ID NO: 19 - DNA, 3144 bp, Escherichia coli
  CDS (1)..(3144)
  misc_feature (1660)..(1660): N = a, c, g, or t
atgattgaatggattattcgtcgctcggtggcgaaccgttttctggtgctgatgggcgcgttgtttctgagtatctggggcacctggactatcattaacacgccagtggatgcactgccggatctttccgatgtgcaggtgattattaaaaccagctatcccggtcaggcaccgcaaatcgttgaaaatcaggtgacttatccgctaaccaccaccatgttgtcggtgcctggcgcgaagactgtgcgcggtttctcgcagtttggcgactcttatgtgtatgtcattttcgaagatggcaccgatccgtactgggcgcgctcgcgggtgctggagtacctcaaccaggtacagggtaagctgcctgcgggagtcagcgccgagctggggccagatgccacgggtgttggctggatctatgaatatgcactggtggatcgcagcggtaagcacgatctggccgatttacgctcattgcaggactggtttctcaaatatgagctaaaaaccatccctgacgttgcggaagtggcgtcggtgggcggtgtggtgaaagagtatcaggtggttatcgatcctcagcgcctggcgcagtatggcatcagtctcgccgaagtaaaaagcgcgctggatgcttcaaaccaggaagcgggcggttcgtcgatcgaactggcggaagcggaatatatggtgcgcgccagcggctatctgcaaacgctcgacgactttaatcacatcgttttaaaagccagtgaaaatggcgtgcccgtttatctgcgtgatgttgcgaaggtccaggttggcccggagatgcgccggggcattgccgaactgaacggcgaaggcgaagtggcgggcggggtggtgatcctgcgttccggcaaaaacgcccgtgaagtgatcgccgccgtgaaggacaaactggaaacgctgaaaagcagtctgccggaaggcgtggagatagttacaacatacgatcgcagtcagctcattgaccgcgccattgacaacctcagcggcaagttgctggaagagtttattgtggtggcggtagtctgtgcgctgtttctctggcatgtgcgctcggcgctggtggcgattatttcgttgccgctggggttgtgtattgcttttattgtcatgcactttcagggactgaacgccaatattatgtcgttgggtggcattgcgattgccgtcggggcgatggtcgatgccgctatcgtcatgatcgagaatgcgcataaacggctggaagagtggcagcaccagcatcctgacgccacgctggataataaaacgcgctggcaggtgatcaccgatgcatctgttgaagtggggccggcgctgtttatcagtctgctgattatcacgctgtcgtttatcccgattttcaccctggaagggcaggaggggcgtctgtttggcccgctggcgttcaccaaaacctatgcgatggcgggcgcggcgctgctggcgatcgtagtgatcccgatcctgatgggctactggatccgtggcaaaattccgccagaaagcagtaacccgctcaatcgctttttgattcgtgtttatcatccgctgttgctgaaagtactgcactggccgaaaaccacgctgttggtggcagcgctttcggtgctgacggttctctggccgntcaataaagttggcggggaatttttaccgcagatcaatgaaggcgacttgttgtatatgccatcgacgctgccggggatttccgcagcggaggcggcgagtatgctgcaaaaaaccgacaagctgattatgagcgtacctgaagtggcgcgggtatttggcaaaaccgggaaagcggaaaccgccaccgattctgctccgctggaaatggtagaaacgaccattcagcttaagccgcaggatcagtggcggcctggcatgacgatggacaaaatcattgaggaactggataacaccgtgcgtctgccggggctggcgaatctgtgggtgccgccaattcgtaaccgtatcgatatgctctcgactggcattaaaagccctatcggcattaaagtttccggcactgtgctggcggatatcgacacgatggcggagcaaattgaagaagtggcgcgaacggtgccaggcgtggcttctgcgcttgctgagcgtctggaaggtgggcgctatatcaacgttgagattaaccgtgaaaaagccgcgcgttacggtatgacggtggcggatgtgcagttgtttgtgacttctgcggtgggcggggcgatggttggcgaaacggtggaagggattgctcgttatccaattaatctgcgttatccgcaaagctggcgcgatagcccgcaggcactgcgccagttgccgatcctcacgccgatgaagcagcaaatcactctggcggacgtggccgacgttaaagtctctaccggaccgtcgatgctgaaaaccgagaatgcgcgcccgacgagctggatttatatcgatgcccgcgatcgtgacatggtgtcggtggttcacgatctgcaaaaagcgatagctgaaaaagtgcagttaaaaccgggcaccagcgtggcattctccgggcagttcgagctactggagcgcgccaaccataagctgaaactgatggtgccgatgacgctgatgatcatcttcgtgctgttgtatctggcgttccgtcgggtgggcgaagcgttgctgattatcagcagcgtaccgtttgcgctggtgggcggcatctggttgctgtggtggatgggctttcatctttccgtggcgacgggcaccggttttattgcccttgccggggtcgccgccgaatttggcgtggtgatgctgatgtatttacgtcacgccatagaggccgagccgtcgttgaataatccgcaaacattcagcgagcagaagctggatgaggcgttatatcacggcgcggtactgcgcgtgcgcccgaaagcgatgacggtggcggtgattatcgctggtctgctgccgattctgtggggaacaggagcagggtcagaggtgatgagccggattgccgcgccgatgattggcggcatgatcaccgcgcctttgctgtcgctgtttattatcccggcggcgtataagctgatgtggctgcaccgacatcgggtacggaaataa

SEQ ID NO: 20 - protein, 1047 aa, Escherichia coli
  misc_feature (554)..(554): The ′Xaa′ at location 554 stands for Ile, Val, Leu, or Phe.
MIEWIIRRSVANRFLVLMGALFLSIWGTWTIINTPVDALPDLSDVQVIIKTSYPGQAPQIVENQVTYPLTTTMLSVPGAKTVRGFSQFGDSYVYVIFEDGTDPYWARSRVLEYLNQVQGKLPAGVSAELGPDATGVGWIYEYALVDRSGKHDLADLRSLQDWFLKYELKTIPDVAEVASVGGVVKEYQVVIDPQRLAQYGISLAEVKSALDASNQEAGGSSIELAEAEYMVRASGYLQTLDDFNHIVLKASENGVPVYLRDVAKVQVGPEMRRGIAELNGEGEVAGGVVILRSGKNAREVIAAVKDKLETLKSSLPEGVEIVTTYDRSQLIDRAIDNLSGKLLEEFIVVAVVCALFLWHVRSALVAIISLPLGLCIAFIVMHFQGLNANIMSLGGIAIAVGAMVDAAIVMIENAHKRLEEWQHQHPDATLDNKTRWQVITDASVEVGPALFISLLIITLSFIPIFTLEGQEGRLFGPLAFTKTYAMAGAALLAIVVIPILMGYWIRGKIPPESSNPLNRFLIRVYHPLLLKVLHWPKTTLLVAALSVLTVLWPXNKVGGEFLPQINEGDLLYMPSTLPGISAAEAASMLQKTDKLIMSVPEVARVFGKTGKAETATDSAPLEMVETTIQLKPQDQWRPGMTMDKIIEELDNTVRLPGLANLWVPPIRNRIDMLSTGIKSPIGIKVSGTVLADIDTMAEQIEEVARTVPGVASALAERLEGGRYINVEINREKAARYGMTVADVQLFVTSAVGGAMVGETVEGIARYPINLRYPQSWRDSPQALRQLPILTPMKQQITLADVADVKVSTGPSMLKTENARPTSWIYIDARDRDMVSVVHDLQKAIAEKVQLKPGTSVAFSGQFELLERANHKLKLMVPMTLMIIFVLLYLAFRRVGEALLIISSVPFALVGGIWLLWWMGFHLSVATGTGFIALAGVAAEFGVVMLMYLRHAIEAEPSLNNPQTFSEQKLDEALYHGAVLRVRPKAMTVAVIIAGLLPILWGTGAGSEVMSRIAAPMIGGMITAPLLSLFIIPAAYKLMWLHRHRVRK

SEQ ID NO: 21 - DNA, 1134 bp, Escherichia coli
  CDS (223)..(1044)
tccgcgaactggcgcatcgcttcggcgttgaaatgccaataaccgaggaaatttatcaagtattatattgcggaaaaaacgcgcgcgaggcagcattgactttactaggtcgtgcacgcaaggacgagcgcagcagccactaaccccagggaacctttgttaccgctatgacccggcccgcgcagaacgggccggtcattatctcatcgtgtggagtaagcaatgtcgtgtgaagaactggaaattgtctggaacaatattaaagccgaagccagaacgctggcggactgtgagccaatgctggccagtttttaccacgcgacgctactcaagcacgaaaaccttggcagtgcactgagctacatgctggcgaacaagctgtcatcgccaattatgcctgctattgctatccgtgaagtggtggaagaagcctacgccgctgacccggaaatgatcgcctctgcggcctgtgatattcaggcggtgcgtacccgcgacccggcagtcgataaatactcaaccccgttgttatacctgaagggttttcatgccttgcaggcctatcgcatcggtcactggttgtggaatcaggggcgtcgcgcactggcaatctttctgcaaaaccaggtttctgtgacgttccaggtcgatattcacccggcagcaaaaattggtcgcggtatcatgcttgaccacgcgacaggcatcgtcgttggtgaaacggcggtgattgaaaacgacgtatcgattctgcaatctgtgacgcttggcggtacgggtaaatctggtggtgaccgtcacccgaaaattcgtgaaggtgtgatgattggcgcgggcgcgaaaatcctcggcaatattgaagttgggcgcggcgcgaagattggcgcaggttccgtggtgctgcaaccggtgccgccgcataccaccgccgctggcgttccggctcgtattgtcggtaaaccagacagcgataagccatcaatggatatggaccagcatttcaacggtattaaccatacatttgagtatggggatgggatctaatgtcctgtgatcgtgccggatgcgatgtaatcatctatccggcctacagtaactaatctctcaataccgctcccgataccccaactgtcg

SEQ ID NO: 22 - protein, 273 aa, Escherichia coli
MSCEELEIVWNNIKAEARTLADCEPMLASFYHATLLKHENLGSALSYMLANKLSSPIMPAIAIREVVEEAYAADPEMIASAACDIQAVRTRDPAVDKYSTPLLYLKGFHALQAYRIGHWLWNQGRRALAIFLQNQVSVTFQVDIHPAAKIGRGIMLDHATGIVVGETAVIENDVSILQSVTLGGTGKSGGDRHPKIREGVMIGAGAKILGNIEVGRGAKIGAGSVVLQPVPPHTTAAGVPARIVGKPDSDKPSMDMDQHFNGINHTFEYGDGI